SEQUENCE LISTING (7 sequences)

SEQ ID NO: 1 - DNA, 4443 bp, Artificial sequence
  Synthetic sequence altered from human sequence
atgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcatcagaaagttcagcattgtgcagaagacccccctgcagatgaatggcattgaggaggactctgatgagcccctggagagaagactgagcctggtgcctgactctgagcagggggaggccatcctgcccagaatctctgtgatcagcacaggccccaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagacagaggaggaggtgcaggacaccagactgtga

SEQ ID NO: 2 - DNA, 4443 bp, Artificial sequence
  Synthetic sequence altered from human sequence
atgcagagaagccccctggagaaggcctctgtggtgagcaagctgttcttcagctggaccagacccatcctgagaaagggctacagacagagactggagctgtctgacatctaccagatcccctctgtggactctgctgacaacctgtctgagaagctggagagagagtgggacagagagctggccagcaagaagaaccccaagctgatcaatgccctgagaagatgcttcttctggagattcatgttctatggcatcttcctgtacctgggggaggtgaccaaggctgtgcagcccctgctgctgggcagaatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggctggcctgggcagaatgatgatgaagtacagagaccagagagctggcaagatctctgagagactggtgatcacctctgagatgattgagaacatccagtctgtgaaggcctactgctgggaggaggccatggagaagatgattgagaacctgagacagacagagctgaagctgaccagaaaggctgcctatgtgagatacttcaacagctctgccttcttcttctctggcttctttgtggtgttcctgtctgtgctgccctatgccctgatcaagggcatcatcctgagaaagatcttcaccaccatcagcttctgcattgtgctgagaatggctgtgaccagacagttcccctgggctgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggggagctgtttgagaaggccaagcagaacaacaacaacagaaagaccagcaatggggatgacagcctgttcttcagcaacttcagcctgctgggcacccctgtgctgaaggacatcaacttcaagattgagagaggccagctgctggctgtggctggcagcacaggggctggcaagaccagcctgctgatgatgatcatgggggagctggagccctctgagggcaagatcaagcactctggcagaatcagcttctgcagccagttcagctggatcatgcctggcaccatcaaggagaacatcatctttggggtgagctatgatgagtacagatacagatctgtgatcaaggcctgccagctggaggaggacatcagcaagtttgctgagaaggacaacattgtgctgggggaggggggcatcaccctgtctgggggccagagagccagaatcagcctggccagagctgtgtacaaggatgctgacctgtacctgctggacagcccctttggctacctggatgtgctgacagagaaggagatctttgagagctgtgtgtgcaagctgatggccaacaagaccagaatcctggtgaccagcaagatggagcacctgaagaaggctgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacagattcagcctggagggggatgcccctgtgagctggacagagaccaagaagcagagcttcaagcagacaggggagtttggggagaagagaaagaacagcatcctgaaccccatcaacagcatcagaaagttcagcattgtgcagaagacccccctgcagatgaatggcattgaggaggactctgatgagcccctggagagaagactgagcctggtgcctgactctgagcagggggaggccatcctgcccagaatctctgtgatcagcacaggccccaccctgcaggccagaagaagacagtctgtgctgaacctgatgacccactctgtgaaccagggccagaacatccacagaaagaccacagccagcaccagaaaggtgagcctggccccccaggccaacctgacagagctggacatctacagcagaagactgagccaggagacaggcctggagatctctgaggagatcaatgaggaggacctgaaggagtgcttctttgatgacatggagagcatccctgctgtgaccacctggaacacctacctgagatacatcacagtgcacaagagcctgatctttgtgctgatctggtgcctggtgatcttcctggctgaggtggctgccagcctggtggtgctgtggctgctgggcaacacccccctgcaggacaagggcaacagcacccacagcagaaacaacagctatgctgtgatcatcaccagcaccagcagctactatgtgttctacatctatgtgggggtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctgggggcatcctgaacagattcagcaaggacattgccatcctggatgacctgctgcccctgaccatctttgacttcatccagctgctgctgattgtgattggggccattgctgtggtggctgtgctgcagccctacatctttgtggccacagtgcctgtgattgtggccttcatcatgctgagagcctacttcctgcagaccagccagcagctgaagcagctggagtctgagggcagaagccccatcttcacccacctggtgaccagcctgaagggcctgtggaccctgagagcctttggcagacagccctactttgagaccctgttccacaaggccctgaacctgcacacagccaactggttcctgtacctgagcaccctgagatggttccagatgagaattgagatgatctttgtgatcttcttcattgctgtgaccttcatcagcatcctgaccacaggggagggggagggcagagtgggcatcatcctgaccctggccatgaacatcatgagcaccctgcagtgggctgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctgggggccagatgacagtgaaggacctgacagccaagtacacagaggggggcaatgccatcctggagaacatcagcttcagcatcagccctggccagagagtgggcctgctgggcagaacaggctctggcaagagcaccctgctgtctgccttcctgagactgctgaacacagagggggagatccagattgatggggtgagctgggacagcatcaccctgcagcagtggagaaaggcctttggggtgatcccccagaaggtgttcatcttctctggcaccttcagaaagaacctggacccctatgagcagtggtctgaccaggagatctggaaggtggctgatgaggtgggcctgagatctgtgattgagcagttccctggcaagctggactttgtgctggtggatgggggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgctgactgcacagtgatcctgtgtgagcacagaattgaggccatgctggagtgccagcagttcctggtgattgaggagaacaaggtgagacagtatgacagcatccagaagctgctgaatgagagaagcctgttcagacaggccatcagcccctctgacagagtgaagctgttcccccacagaaacagcagcaagtgcaagagcaagccccagattgctgccctgaaggaggagaccgaggaggaggtgcaggacaccagactgtga

SEQ ID NO: 3 - RNA, 4443 nt, Artificial sequence
  Synthetic sequence altered from human sequence
augcagagaagcccccuggagaaggccucuguggugagcaagcuguucuucagcuggaccagacccauccugagaaagggcuacagacagagacuggagcugucugacaucuaccagauccccucuguggacucugcugacaaccugucugagaagcuggagagagagugggacagagagcuggccagcaagaagaaccccaagcugaucaaugcccugagaagaugcuucuucuggagauucauguucuauggcaucuuccuguaccugggggaggugaccaaggcugugcagccccugcugcugggcagaaucauugccagcuaugacccugacaacaaggaggagagaagcauugccaucuaccugggcauuggccugugccugcuguucauugugagaacccugcugcugcacccugccaucuuuggccugcaccacauuggcaugcagaugagaauugccauguucagccugaucuacaagaagacccugaagcugagcagcagagugcuggacaagaucagcauuggccagcuggugagccugcugagcaacaaccugaacaaguuugaugagggccuggcccuggcccacuuuguguggauugccccccugcagguggcccugcugaugggccugaucugggagcugcugcaggccucugccuucuguggccugggcuuccugauugugcuggcccuguuccaggcuggccugggcagaaugaugaugaaguacagagaccagagagcuggcaagaucucugagagacuggugaucaccucugagaugauugagaacauccagucugugaaggccuacugcugggaggaggccauggagaagaugauugagaaccugagacagacagagcugaagcugaccagaaaggcugccuaugugagauacuucaacagcucugccuucuucuucucuggcuucuuugugguguuccugucugugcugcccuaugcccugaucaagggcaucauccugagaaagaucuucaccaccaucagcuucugcauugugcugagaauggcugugaccagacaguuccccugggcugugcagaccugguaugacagccugggggccaucaacaagauccaggacuuccugcagaagcaggaguacaagacccuggaguacaaccugaccaccacagagguggugauggagaaugugacagccuucugggaggagggcuuuggggagcuguuugagaaggccaagcagaacaacaacaacagaaagaccagcaauggggaugacagccuguucuucagcaacuucagccugcugggcaccccugugcugaaggacaucaacuucaagauugagagaggccagcugcuggcuguggcuggcagcacaggggcuggcaagaccagccugcugaugaugaucaugggggagcuggagcccucugagggcaagaucaagcacucuggcagaaucagcuucugcagccaguucagcuggaucaugccuggcaccaucaaggagaacaucaucuuuggggugagcuaugaugaguacagauacagaucugugaucaaggccugccagcuggaggaggacaucagcaaguuugcugagaaggacaacauugugcugggggaggggggcaucacccugucugggggccagagagccagaaucagccuggccagagcuguguacaaggaugcugaccuguaccugcuggacagccccuuuggcuaccuggaugugcugacagagaaggagaucuuugagagcugugugugcaagcugauggccaacaagaccagaauccuggugaccagcaagauggagcaccugaagaaggcugacaagauccugauccugcaugagggcagcagcuacuucuauggcaccuucucugagcugcagaaccugcagccugacuucagcagcaagcugaugggcugugacagcuuugaccaguucucugcugagagaagaaacagcauccugacagagacccugcacagauucagccuggagggggaugccccugugagcuggacagagaccaagaagcagagcuucaagcagacaggggaguuuggggagaagagaaagaacagcauccugaaccccaucaacagcaucagaaaguucagcauugugcagaagaccccccugcagaugaauggcauugaggaggacucugaugagccccuggagagaagacugagccuggugccugacucugagcagggggaggccauccugcccagaaucucugugaucagcacaggccccacccugcaggccagaagaagacagucugugcugaaccugaugacccacucugugaaccagggccagaacauccacagaaagaccacagccagcaccagaaaggugagccuggccccccaggccaaccugacagagcuggacaucuacagcagaagacugagccaggagacaggccuggagaucucugaggagaucaaugaggaggaccugaaggagugcuucuuugaugacauggagagcaucccugcugugaccaccuggaacaccuaccugagauacaucacagugcacaagagccugaucuuugugcugaucuggugccuggugaucuuccuggcugagguggcugccagccugguggugcuguggcugcugggcaacaccccccugcaggacaagggcaacagcacccacagcagaaacaacagcuaugcugugaucaucaccagcaccagcagcuacuauguguucuacaucuauguggggguggcugacacccugcuggccaugggcuucuucagaggccugccccuggugcacacccugaucacagugagcaagauccugcaccacaagaugcugcacucugugcugcaggcccccaugagcacccugaacacccugaaggcugggggcauccugaacagauucagcaaggacauugccauccuggaugaccugcugccccugaccaucuuugacuucauccagcugcugcugauugugauuggggccauugcugugguggcugugcugcagcccuacaucuuuguggccacagugccugugauuguggccuucaucaugcugagagccuacuuccugcagaccagccagcagcugaagcagcuggagucugagggcagaagccccaucuucacccaccuggugaccagccugaagggccuguggacccugagagccuuuggcagacagcccuacuuugagacccuguuccacaaggcccugaaccugcacacagccaacugguuccuguaccugagcacccugagaugguuccagaugagaauugagaugaucuuugugaucuucuucauugcugugaccuucaucagcauccugaccacaggggagggggagggcagagugggcaucauccugacccuggccaugaacaucaugagcacccugcagugggcugugaacagcagcauugauguggacagccugaugagaucugugagcagaguguucaaguucauugacaugcccacagagggcaagcccaccaagagcaccaagcccuacaagaauggccagcugagcaaggugaugaucauugagaacagccaugugaagaaggaugacaucuggcccucugggggccagaugacagugaaggaccugacagccaaguacacagaggggggcaaugccauccuggagaacaucagcuucagcaucagcccuggccagagagugggccugcugggcagaacaggcucuggcaagagcacccugcugucugccuuccugagacugcugaacacagagggggagauccagauugauggggugagcugggacagcaucacccugcagcaguggagaaaggccuuuggggugaucccccagaagguguucaucuucucuggcaccuucagaaagaaccuggaccccuaugagcaguggucugaccaggagaucuggaagguggcugaugaggugggccugagaucugugauugagcaguucccuggcaagcuggacuuugugcugguggaugggggcugugugcugagccauggccacaagcagcugaugugccuggccagaucugugcugagcaaggccaagauccugcugcuggaugagcccucugcccaccuggacccugugaccuaccagaucaucagaagaacccugaagcaggccuuugcugacugcacagugauccugugugagcacagaauugaggccaugcuggagugccagcaguuccuggugauugaggagaacaaggugagacaguaugacagcauccagaagcugcugaaugagagaagccuguucagacaggccaucagccccucugacagagugaagcuguucccccacagaaacagcagcaagugcaagagcaagccccagauugcugcccugaaggaggagacagaggaggaggugcaggacaccagacuguga

SEQ ID NO: 4 - RNA, 4443 nt, Artificial sequence
  Synthetic sequence altered from human sequence
augcagagaagcccccuggagaaggccucuguggugagcaagcuguucuucagcuggaccagacccauccugagaaagggcuacagacagagacuggagcugucugacaucuaccagauccccucuguggacucugcugacaaccugucugagaagcuggagagagagugggacagagagcuggccagcaagaagaaccccaagcugaucaaugcccugagaagaugcuucuucuggagauucauguucuauggcaucuuccuguaccugggggaggugaccaaggcugugcagccccugcugcugggcagaaucauugccagcuaugacccugacaacaaggaggagagaagcauugccaucuaccugggcauuggccugugccugcuguucauugugagaacccugcugcugcacccugccaucuuuggccugcaccacauuggcaugcagaugagaauugccauguucagccugaucuacaagaagacccugaagcugagcagcagagugcuggacaagaucagcauuggccagcuggugagccugcugagcaacaaccugaacaaguuugaugagggccuggcccuggcccacuuuguguggauugccccccugcagguggcccugcugaugggccugaucugggagcugcugcaggccucugccuucuguggccugggcuuccugauugugcuggcccuguuccaggcuggccugggcagaaugaugaugaaguacagagaccagagagcuggcaagaucucugagagacuggugaucaccucugagaugauugagaacauccagucugugaaggccuacugcugggaggaggccauggagaagaugauugagaaccugagacagacagagcugaagcugaccagaaaggcugccuaugugagauacuucaacagcucugccuucuucuucucuggcuucuuugugguguuccugucugugcugcccuaugcccugaucaagggcaucauccugagaaagaucuucaccaccaucagcuucugcauugugcugagaauggcugugaccagacaguuccccugggcugugcagaccugguaugacagccugggggccaucaacaagauccaggacuuccugcagaagcaggaguacaagacccuggaguacaaccugaccaccacagagguggugauggagaaugugacagccuucugggaggagggcuuuggggagcuguuugagaaggccaagcagaacaacaacaacagaaagaccagcaauggggaugacagccuguucuucagcaacuucagccugcugggcaccccugugcugaaggacaucaacuucaagauugagagaggccagcugcuggcuguggcuggcagcacaggggcuggcaagaccagccugcugaugaugaucaugggggagcuggagcccucugagggcaagaucaagcacucuggcagaaucagcuucugcagccaguucagcuggaucaugccuggcaccaucaaggagaacaucaucuuuggggugagcuaugaugaguacagauacagaucugugaucaaggccugccagcuggaggaggacaucagcaaguuugcugagaaggacaacauugugcugggggaggggggcaucacccugucugggggccagagagccagaaucagccuggccagagcuguguacaaggaugcugaccuguaccugcuggacagccccuuuggcuaccuggaugugcugacagagaaggagaucuuugagagcugugugugcaagcugauggccaacaagaccagaauccuggugaccagcaagauggagcaccugaagaaggcugacaagauccugauccugcaugagggcagcagcuacuucuauggcaccuucucugagcugcagaaccugcagccugacuucagcagcaagcugaugggcugugacagcuuugaccaguucucugcugagagaagaaacagcauccugacagagacccugcacagauucagccuggagggggaugccccugugagcuggacagagaccaagaagcagagcuucaagcagacaggggaguuuggggagaagagaaagaacagcauccugaaccccaucaacagcaucagaaaguucagcauugugcagaagaccccccugcagaugaauggcauugaggaggacucugaugagccccuggagagaagacugagccuggugccugacucugagcagggggaggccauccugcccagaaucucugugaucagcacaggccccacccugcaggccagaagaagacagucugugcugaaccugaugacccacucugugaaccagggccagaacauccacagaaagaccacagccagcaccagaaaggugagccuggccccccaggccaaccugacagagcuggacaucuacagcagaagacugagccaggagacaggccuggagaucucugaggagaucaaugaggaggaccugaaggagugcuucuuugaugacauggagagcaucccugcugugaccaccuggaacaccuaccugagauacaucacagugcacaagagccugaucuuugugcugaucuggugccuggugaucuuccuggcugagguggcugccagccugguggugcuguggcugcugggcaacaccccccugcaggacaagggcaacagcacccacagcagaaacaacagcuaugcugugaucaucaccagcaccagcagcuacuauguguucuacaucuauguggggguggcugacacccugcuggccaugggcuucuucagaggccugccccuggugcacacccugaucacagugagcaagauccugcaccacaagaugcugcacucugugcugcaggcccccaugagcacccugaacacccugaaggcugggggcauccugaacagauucagcaaggacauugccauccuggaugaccugcugccccugaccaucuuugacuucauccagcugcugcugauugugauuggggccauugcugugguggcugugcugcagcccuacaucuuuguggccacagugccugugauuguggccuucaucaugcugagagccuacuuccugcagaccagccagcagcugaagcagcuggagucugagggcagaagccccaucuucacccaccuggugaccagccugaagggccuguggacccugagagccuuuggcagacagcccuacuuugagacccuguuccacaaggcccugaaccugcacacagccaacugguuccuguaccugagcacccugagaugguuccagaugagaauugagaugaucuuugugaucuucuucauugcugugaccuucaucagcauccugaccacaggggagggggagggcagagugggcaucauccugacccuggccaugaacaucaugagcacccugcagugggcugugaacagcagcauugauguggacagccugaugagaucugugagcagaguguucaaguucauugacaugcccacagagggcaagcccaccaagagcaccaagcccuacaagaauggccagcugagcaaggugaugaucauugagaacagccaugugaagaaggaugacaucuggcccucugggggccagaugacagugaaggaccugacagccaaguacacagaggggggcaaugccauccuggagaacaucagcuucagcaucagcccuggccagagagugggccugcugggcagaacaggcucuggcaagagcacccugcugucugccuuccugagacugcugaacacagagggggagauccagauugauggggugagcugggacagcaucacccugcagcaguggagaaaggccuuuggggugaucccccagaagguguucaucuucucuggcaccuucagaaagaaccuggaccccuaugagcaguggucugaccaggagaucuggaagguggcugaugaggugggccugagaucugugauugagcaguucccuggcaagcuggacuuugugcugguggaugggggcugugugcugagccauggccacaagcagcugaugugccuggccagaucugugcugagcaaggccaagauccugcugcuggaugagcccucugcccaccuggacccugugaccuaccagaucaucagaagaacccugaagcaggccuuugcugacugcacagugauccugugugagcacagaauugaggccaugcuggagugccagcaguuccuggugauugaggagaacaaggugagacaguaugacagcauccagaagcugcugaaugagagaagccuguucagacaggccaucagccccucugacagagugaagcuguucccccacagaaacagcagcaagugcaagagcaagccccagauugcugcccugaaggaggagaccgaggaggaggugcaggacaccagacuguga

SEQ ID NO: 5 - DNA, 4443 bp, Homo sapiens
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgaatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttag

SEQ ID NO: 6 - DNA, 56 bp, Homo sapiens
aataaacaaaatacaggatttcttacaaaagcaagaatataagacattggaatata

SEQ ID NO: 7 - DNA, 87 bp, Homo sapiens
aataaacaaaatacaggtaatgtaccataatgctgcattatatactatgatttaaataatcagtcaatagatcagttctaatgaact

(19) United States
(12) Patent Application Publication
(10) Pub. No.: US 2011/0035819 A1
(43) Pub. Date: Feb. 10, 2011

(54) CODON OPTIMIZED CFTR

(75) Inventors: Mark J. Cooper, Moreland Hills, OH (US); Linas Padegimas, Mayfield Heights, OH (US)

Correspondence Address: BANNER & WITCOFF, LTD., 1100 13th STREET, N.W., SUITE 1200, WASHINGTON, DC, 20005-4051, US

(73) Assignee: COPERNICUS THERAPEUTICS INC., Cleveland, OH (US)

(21) Appl. No.: 12/443,933

(22) PCT Filed: Oct. 12, 2007
(86) PCT No.: PCT/US07/21862

§ 371 Date: Aug. 18, 2009

Related U.S. Application Data
(60) Provisional application No. 60/851,055, filed on Oct. 12, 2006.
(60) Provisional application No. 60/885,827, filed on Jan. 19, 2007.
(60) Provisional application No. 60/907,852, filed on Apr. 19, 2007.

(51) Int. Cl. A01K 67/027; C07H 21/04; C12N 15/63; C12N 5/10; C12P 21/00; A61K 48/00; C12N 15/32; C12N 5/00

(52) U.S. Cl. 800/14; 536/23.1; 435/320.1; 435/325; 435/69.1; 514/44 R; 800/21; 435/375

(57) ABSTRACT

A synthetic hCFTR DNA sequence has been developed that produces remarkably high levels of hCFTR mRNA and protein in dosed murine lungs and human cells in culture compared to the natural hCFTR cDNA. This synthetic DNA addresses problems inherent in some natural cDNAs, such as premature transcriptional truncation sites introduced during cDNA synthesis. Introns are initially present in mRNA until the mRNA is processed. cDNA made from processed mRNA is devoid of introns. Thus DNA sequences (exon junctions) are present in a cDNA molecule which are not present in cells in nature. These exon junctions may affect transcription. Methods for improving expression of CFTR are based on sequence changes in cDNA molecules. The improvement methods may be applied to other cDNA molecules which are refractory to in vivo expression efforts. Compositions embodying the sequence changes increase the production of both transgenic mRNA and protein from cDNA molecules.

This application claims the benefit of provisional application Ser. No. 60/851,055 filed Oct. 12, 2006, Ser. No. 60/885,827 filed Jan. 19, 2007, and Ser. No. 60/907,852 filed Apr. 19, 2007. The disclosures of each are expressly incorporated herein.

### TECHNICAL FIELD OF THE INVENTION

This invention is related to the area of Cystic Fibrosis. In particular, it relates to the area of gene therapy vectors for Cystic Fibrosis and other diseases.

### BACKGROUND OF THE INVENTION

Cystic fibrosis is caused by various mutations in the cystic fibrosis transmembrane conductance regulator (CFTR) gene, a membrane-bound chloride channel. Mutations in CF results in thick, inspisated pulmonary mucus, which results in recurrent lung infections, subsequent structural lung damage, and eventual respiratory failure. Patients with CF also develop other manifestations due to blockage of ducts by thick secretions, including insufficient release of pancreatic digestive enzymes and insulin, resulting in malnutrition and diabetes. Most CF patients require ingestion of pancreatic enzyme supplements. The average survival of CF patients is in the mid 30s.

To address the pulmonary manifestations of CF, it is generally believed that one needs to obtain expression of hCFTR (human CFTR) in the proximal lung epithelium, and possibly in the proximal ductal epithelium, as well. An expression level of an exogenous CFTR gene at 10% of the endogenous CFTR mRNA level may be therapeutic. See Davis, P. B., Centennial Review, Am. J. Respir. Crit. Care Med., 173: 475-482, 2006. This assessment is based on the level of CFTR mRNA in various sub-populations of CF carriers who have low levels of CFTR mRNA but are asymptomatic. Other supportive data for this estimate include measurements in tissue culture models and evidence of electrical correction of the Cl⁻ channel defect if normal CFTR mRNA is on the order of 6-10%.

In most patients, the CF airways are covered with thick mucus, which may impede effective gene transfer to the underlying epithelial cells of the lung. Thus prospective gene transfer systems that address such physiologic barriers may be important for effective CF gene therapy. Previous studies have demonstrated that nanoparticle vectors effectively transfect respiratory epithelial cells in the nares of CF subjects [Konstan M W, Davis P B, Wagener J S, Hilliard K A, Stern R C, Milgram L J, Kowalczyk T H, Hyatt S L, Fink T L, Gedeon C R, Oette S M, Payne J M, Muhammad O, Ziady A G, Moen R C, Cooper M J. “Compacted DNA nanoparticles administered to the nasal mucosa of cystic fibrosis subjects are safe and demonstrate partial to complete cystic fibrosis transmembrane regulator reconstitution.” Hum Gene Ther. 2004 December; 15 (12):1255-69.]

Initial phase I intranasal clinical trials have been concluded and provided encouraging results: no adverse events were attributed to the DNA nanoparticles and 8/12 subjects demonstrated improved CFTR chloride channel function as assessed by nasal potential difference measurements. In addition, 4/12 subjects had nasal potential difference (NPD) values within the normal range [Konstan et al, supra].

There is a continuing need in the art to improve existing vectors so that meaningful levels of CFTR expression can be achieved to provide clinically measurable improvements.

### SUMMARY OF THE INVENTION

According to one embodiment of the invention a composition is provided that comprises a nucleic acid molecule comprising a sequence as shown in SEQ ID NO: 1 or 2 (DNA) or SEQ ID NO: 3 or 4 (RNA).

According to another embodiment a method is provided for producing hCFTR-encoding mRNA and hCFTR protein. A composition comprising a nucleic acid molecule comprising a sequence as shown in SEQ ID NO: 1 or 2 (DNA) or SEQ ID NO: 3 or 4 (RNA) is introduced into mammalian cells. The sequence can be operably linked to expression control sequences. The cells express hCFTR-encoding mRNA and hCFTR protein as a result of the introduction.

According to yet another embodiment of the invention a method is provided for producing hCFTR-encoding mRNA and hCFTR protein. A composition comprising a nucleic acid molecule comprising a sequence as shown in SEQ ID NO: 1 or 2 (DNA) or SEQ ID NO: 3 or 4 (RNA) is introduced into human lung cells in a human Cystic Fibrosis patient via an aerosol. The sequence can be operably linked to expression control sequences. The nucleic acid molecule is compacted in particles with a polycation; the particles are unimolecular with respect to nucleic acid. As a result of the introduction, the cells express hCFTR-encoding mRNA and hCFTR protein.

An additional embodiment of the invention provides a method to increase the expression of an mRNA or protein from a cDNA molecule. The cDNA molecule is inspected to ascertain the presence of a premature transcription termination signal. The premature transcription termination signal of the cDNA molecule is eliminated without altering its encoded amino acid sequence, thereby forming a cDNA molecule with an altered sequence. The cDNA molecule with the altered sequence is introduced into a cell where it is expressed.

These and other embodiments which will be apparent to those of skill in the art upon reading the specification provide the art with improved methods and reagents for gene therapy and protein expression.

### BRIEF DESCRIPTION OF THE DRAWINGS

FIG. 1. Plots of hCFTR/mCFTR mRNA levels in mouse lungs dosed intranasally with compacted pCMVCFTR, pUCF, and pUCF2 plasmids. pCMVCFTR and pUCF both contain the identical “natural” CFTR cDNA, whereas pUCF2 contains a codon-optimized, CpG-depleted (except 1 CpG in the 3′ terminus), and 5′ and 3′ UTR truncated in vitro synthesized DNA. Two days and 14 days after dosing, lungs were harvested for qRT-PCR analysis of hCFTR mRNA expression, tabulated as a hCFTR/mCFTR ratio. The only statistically significant differences (Mann-Whitney test) were between day 14 pUCF2 and pCMVCFTR (p₂=0.032), and day 14 pUCF2 and pUCF (p₂=0.040). All other pairwise comparisons, including day 2 pCMVCFTR and pUCF2, were not significant. N=9/group.

FIG. 2. IP/Western blot analysis of hCFTR expression in HEK293 cells 2 days after transfection with pUCF, pUCF2, or control pCMVCFTR plasmid. Cells were transfected with lipofectamine and either low (L, 0.75 ug) or high (H, 3 ug) amounts of CFTR plasmid. Luc, cells transfected with luciferase plasmid. NT, non-transfected. Anti-CFTR monoclonal antibody 1660 (R&D Systems), directed against R domain codons 590-830, was used in both IP and detection protocols.

FIG. 3 ELISA assay for hCFTR. Standard curve using HEK293 cells transfected with pUCF2.

FIG. 4. Plots of hCFTR/mCFTR mRNA levels at days 2 and 14 in mouse lungs dosed with compacted pUCF22; data for pCMVCFTR, pUCF, and pUCF2 are included for comparison. pCMVCFTR and pUCF both contain the identical ‘natural’ CFTR cDNA, whereas pUCF2 contains a codon-optimized, CpG-depleted (except 1 CpG in the 3′ terminus), and 5′ and 3′ UTR truncated in vitro synthesized DNA. pUCF22 contains the CO-CFTR of pUCF2 but is completely CpG depleted. In addition, pUCF22 contains the R6K ori, Km^{R}, and Km promoter. In contrast to the other groups which received 100 μg doses intranasally, pUCF22 mice received 200 μg DNA by the intratracheal route. As expected, there were no ‘missed doses’ in this group, and the variances per group are smaller than for intranasal dosings. N=6/time point.

FIG. 5. Primers and probes used to amplify hCFTR transcripts from pCMVCFTR, pUCF, and pUCF2. Sets G, L, and M detect appropriately spliced transcripts, and 3′ sets C, J, and K have been used previously (FIG. 1, 4) to detect full-length hCFTR mRNA generated from these plasmids.

FIG. 6. Proposed classes of hCFTR transcripts produced by pCMVCFTR, pUCF, and pUCF2.

FIG. 7A-7C. Transcriptional termination sequences. (FIG. 7A) Typical eukaryotic transcriptional termination sequences includes ‘AAUAAA’ followed by a uridine rich element (URE), with a preferred cleavage site (A>U>C>>G) between these two motifs [2]. Spacing between elements is shown. (FIG. 7B) hCFTR cDNA (SEQ ID NO: 6) ‘AATAAA’ sequence has a URE element beginning at +14 bp, with a potential cleavage site ‘A’ at +12 (shown in bold). This URE is closer than typical to the ‘AAUAAA’ site, although there is considerable variability of the URE spacing interval [2]. (FIG. 7C) hCFTR genomic sequence (SEQ ID NO: 7) has ‘AATAAA’ motif in exon 7, which is soon followed by intron 7 (in bold). No candidate URE sequence is observed in the next 81 bp.

FIG. 8. Recombinant constructions used in the present study are graphically depicted: pUL, pUCF, pUCF22, pCMVCFTR, and pUCF2. Lollipops represent CpG dinucleotides.

FIG. 9. Comparison of natural cDNA to synthetic DNA for CFTR with 1 CpG (SEQ ID NO: 5 and 2, respectively.

### DETAILED DESCRIPTION OF THE INVENTION

The present inventors have developed a synthetic CFTR DNA segment (‘CO-CFTR’; SEQ ID NO: 1 and 2) that produces improves mRNA and protein levels in the mouse lung (mRNA) and cells (protein) compared to “natural” hCFTR cDNA (i.e., cDNA made from native mRNA; SEQ ID NO: 5). This synthetic CFTR segment is codon optimized (hence the ‘CO’ label), CpG depleted, removes endogenous 5′ and 3′ UTRs, and has an optimized Kozak sequence. One version has one C-terminal CpG island (SEQ ID NO: 2), and another version has no CpG islands (SEQ ID NO: 1). Quantitative RT-PCR data demonstrate 35-fold increase in CFTR mRNA compared to “natural” cDNA in the mouse lung. Immunoprecipitation Western blots show 9-fold improvement in CFTR protein compared to “natural” cDNA. Human CFTR ELISA (enzyme-linked immunoadsorbent assay) in transfected HEK293 (human kidney) cells show an ˜19-fold increase in hCFTR protein compared to an analogous plasmid encoding “natural” hCFTR cDNA.

Because of the many differences between natural and synthetic hCFTR cDNA it is not known whether one or more of the changes contribute to or are responsible for the improved expression. Although applicants do not intend to be bound by any theory or mechanism, it is possible that the improvement is due, in part, to the obliteration of a premature transcriptional terminator in the “natural” cDNA. This terminator is not present in genomic DNA, hence it is not relevant in whole animals that are not transgenic. We have identified such a transcriptional terminator in the “natural” cDNA which is not present in the codon-optimized hCFTR DNA.

Nucleic acid compositions according to the present invention may be solid (e.g., lyophilized or precipitated) or liquid or aerosolized. They may be RNA (e.g., SEQ ID NO: 3 and 4) or DNA (SEQ ID NO: 1 and 2). Such compositions may be linear nucleic acid fragments or included in plasmid or viral vectors, whether linear or circular. Many viral vectors are known in the art and they can be selected by the skilled artisan for their known properties. Exemplary vectors are employed in the working examples below, but others can be used as well. Nonviral vectors for cystic fibrosis therapy are discussed in Alton, E.W.F.W., Proceedings of the American Thoracic Society, 1, 296-301, 2004.

Expression control sequences are known in the art and will be typically employed in the invention. These may be used to initiate, promote, or terminate transcription, for example, or to enhance translation. These are operably linked to the coding sequence, i.e., they are within the requisite proximity on a nucleic acid molecule to affect the function. Proper placement for these elements is well known in the art.

Mammalian cells are the typical targets of the human nucleic acid molecules of the invention. These can be in culture, in tissues, in perfused organs, in whole animal models, or in patients or control individuals. In the case of Cystic Fibrosis, typical target cells are lung cells or pancreatic cells. Epithelial and ductal cells of the lung and pancreatic may be particularly targeted. Targeting can be effected by local delivery or installation of the nucleic acids. Other delivery means include intravenous and endoscopic delivery. Other targeted cells may include those of the gastrointestinal tract, of the endocrine system, and any other affected organ or organ system. Delivery can also be performed in utero to a fetus.

The nucleic acid vectors may be delivered by any means known in the art. One way to package and deliver nucleic acids is via compacted nanoparticles. These are typically formed with a polycation, such as polylysine. Nanoparticles can be formed which have a single molecule of nucleic acid.

The results of the experiments below indicate that unsuspected premature termination of transcription from a terminator formed by making a cDNA can hamper expression. Noting such fortuitous terminators and removing them can increase expression. This may be a generally useful method for increasing expression in the class of cDNA molecules that contain such a fortuitous terminator. To ascertain if a terminator is present, the sequence of the cDNA molecule in question is inspected. This may be by eye/hand/human brain, or by machine/computer. The features of transcription termination signals are well known in the art. Following ‘AAUAAA’ there typically is a uridine rich element (URE). The cleavage site typically occurs 11 to 24 bases downstream of ‘AAUAAA’, and the URE sequence occurs 10 to 30 bp after the cleavage site. Eliminating the signal involves changing the sequence sufficiently so that it no longer functions as a transcription termination signal. The “eliminated” cDNA is then introduced into cells so that it can drive expression of mRNA and/or protein.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

### Evaluation of Codon-Optimized hCFTR cDNA

Since codon-optimization of the hCFTR sequence might improve translation efficiency in transfected lung cells, the following hCFTR DNA was synthesized. Presented below is an analysis of the natural hCFTR cDNA and a codon-optimized hCFTR sequence. The latter also was CpG-island depleted except for a single CpG island in the 3-prime region. In other versions, this single CpG island was removed (no CpG islands)

Natural hCFTR cDNA: CpG islands = 58
Codon Usage Table 1

| Amino Acids | | Ranks (most -> least) | | | | | |
|---|---|---|---|---|---|---|---|
| Single-letter | Three-letter | 1 | 2 | 3 | 4 | 5 | 6 |
| F | Phe | TTC = 38 | TTT = 47 | | | | |
| L | Leu | CTG = 37 | CTC = 24 | CTT = 32 | TTG = 34 | TTA = 37 | CTA = 19 |
| S | Ser | AGC = 24 | TCC = 17 | TCT = 30 | TCA = 29 | AGT = 19 | TCG = 4 |
| Y | Tyr | TAC = 18 | TAT = 22 | | | | |
| * | Ter | TGA = 0 | TAA = 0 | TAG = 1 | | | |
| C | Cys | TGC = 10 | TGT = 8 | | | | |
| W | Trp | TGG = 23 | | | | | |
| P | Pro | CCC = 11 | CCT = 20 | CCA = 13 | CCG = 1 | | |
| H | His | CAC = 14 | CAT = 11 | | | | |
| Q | Gln | CAG = 31 | CAA = 36 | | | | |
| R | Arg | AGA = 37 | AGG = 16 | CGG = 8 | CGC = 5 | CGA = 9 | CGT = 3 |
| I | Ile | ATC = 36 | ATT = 50 | ATA = 33 | | | |
| M | Met | ATG = 38 | | | | | |
| T | Thr | ACC = 15 | ACA = 33 | ACT = 3 | ACG = 32 | | |
| N | Asn | AAC = 29 | AAT = 25 | | | | |
| K | Lys | AAG = 35 | AAA = 57 | | | | |
| V | Val | GTG = 36 | GTC = 18 | GTT = 23 | GTA = 12 | | |
| A | Ala | GCC = 16 | GCT = 27 | GCA = 34 | GCG = 6 | | |
| D | Asp | GAC = 20 | GAT = 38 | | | | |
| E | Glu | GAG = 30 | GAA = 63 | | | | |
| G | Gly | GGC = 15 | GGG = 16 | GGA = 38 | GGT = 15 | | |

| Total | 493 | 988 |
|---|---|---|
| | (33.3%) | (66.7%) |

Codon-Optimized and CpG Island-Depleted hCFTR DNA: CpG islands = 1
Codon Usage Table 2

| Amino Acids | | Ranks (most -> least) | | | | | |
|---|---|---|---|---|---|---|---|
| Single-letter | Three-letter | 1 | 2 | 3 | 4 | 5 | 6 |
| F | Phe | TTC = 63 | TTT = 22 | | | | |
| L | Leu | CTG = 183 | CTC = 0 | CTT = 0 | TTG = 0 | TTA = 0 | CTA = 0 |
| S | Ser | AGC = 87 | TCC = 0 | TCT = 36 | TCA = 0 | AGT = 0 | TCG = 0 |
| Y | Tyr | TAC = 18 | TAT = 22 | | | | |
| * | Ter | TGA = 1 | TAA = 0 | TAG = 0 | | | |
| C | Cys | TGC = 13 | TGT = 5 | | | | |
| W | Trp | TGG = 23 | | | | | |
| P | Pro | CCC = 33 | CCT = 12 | CCA = 0 | CCG = 0 | | |
| H | His | CAC = 14 | CAT = 11 | | | | |
| Q | Gln | CAG = 67 | CAA = 0 | | | | |
| R | Arg | AGA = 78 | AGG = 0 | CGG = 0 | CGC = 0 | CGA = 0 | CGT = 0 |
| I | Ile | ATC = 88 | ATT = 31 | ATA = 0 | | | |
| M | Met | ATG = 38 | | | | | |
| T | Thr | ACC = 59 | ACA = 24 | ACT = 0 | ACG = 0 | | |
| N | Asn | AAC = 46 | AAT = 8 | | | | |
| K | Lys | AAG = 92 | AAA = 0 | | | | |
| V | Val | GTG = 89 | GTC = 0 | GTT = 0 | GTA = 0 | | |
| A | Ala | GCC = 55 | GCT = 28 | GCA = 0 | GCG = 0 | | |
| D | Asp | GAC = 43 | GAT = 15 | | | | |
| E | Glu | GAG = 93 | GAA = 0 | | | | |
| G | Gly | GGC = 59 | GGG = 25 | GGA = 0 | GGT = | | |

| Total | 1242 | 239 |
|---|---|---|
| | (83.9%) | (16.1%) |

Note that the preferred codon-usage has increased from 33.3% to 83.9%. Note that the pattern of preferred codon usage in humans and mice is very similar (identical in 18 amino acids, with very slight second codon preference for proline and arginine).

### Example 2

### Evaluation of Expression Vectors Encoding Natural hCFTR cDNA

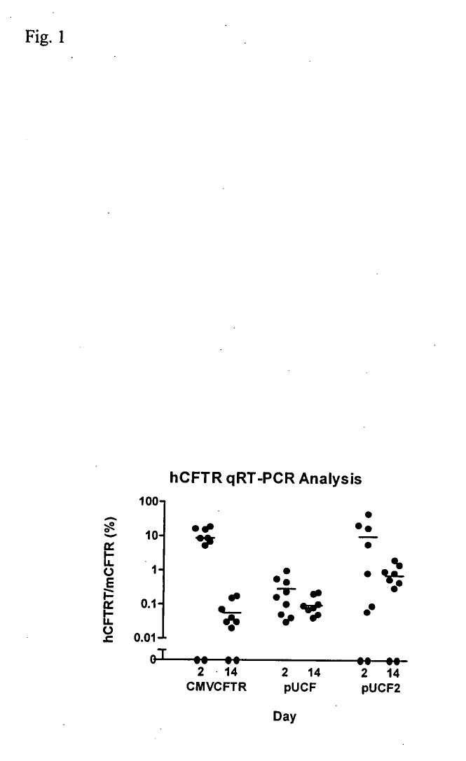

We first evaluated hCFTR mRNA levels in mice dosed with our prior CMVCFTR clinical trial plasmid, with lung harvests at days 2 and 14 (FIG. 1). Of 9 dosed mice, 2 had no hCFTR signal on both days 2 and 14, consistent with ‘missed doses’ in these intranasal (TN) dosed mice or true negatives. The average hCFTR/mCFTR ratio for all data on day 2 was 8.7% (+/−6.7%, SD) and by day 14 this ratio had fallen to 0.06% (+/−0.06%). This study was repeated with a derivative of the pUL plasmid (pUCF) with luciferase replaced by the same CFTR cDNA used in the CMVCFTR plasmid. Although luciferase activity in pUL is comparable to our analogous CMVluc plasmid, hCFTR expression levels were lower than pCMVCFTR on day 2, with a hCFTR/mCFTR ratio of 0.28% and falling to 0.09% by day 14. This result with pUCF was disappointing and could be due to several factors, as discussed below.

It is appreciated that multiple factors may be involved in hCFTR mRNA expression levels, including CpG motifs in CFTR cDNA and their potential influence on expression extinction; promoter transcriptional efficiency; appropriate post-transcriptional CFTR mRNA splicing and its linkage to cytoplasmic transport, post-transcriptional CFTR mRNA stability as linked to secondary structure (which likely would be altered in the process of codon-optimization); and post-transcriptional CFTR mRNA stability as controlled by interactive domains in the 3′ UTR (as recently described by Baudouin-Legros et. al (1)). Regarding protein expression levels, both codon-optimization and presence of a full Kozak consensus sequence are likely important. In an effort to modulate and hopefully improve the influence of some of these factors, we designed a codon-optimized CFTR (CO-CFTR) DNA that has a single CpG in the 3′ terminus and completely lacks the natural 5′ and 3′ UTRs. Table 3 summarizes important properties of pCMVCFTR, pUCF, and pUCF2 (a derivative of pUCF containing CO-CFTR). CO-CFTR has a different mRNA sequence than natural CFTR and this may influence mRNA stability relative to its secondary structure. Of note, both pCMVCFTR and pUCF contain identical CFTR sequences, including 2 potential alternative splice acceptor sites in the first 300 bp of coding sequences, whereas there are no alternative splice acceptor sites in CO-CFTR within this region—a finding that might be important in optimizing appropriate splicing. Also note that there are only minor differences in the size of 5′ and 3′ UTRs in these constructs and the natural CFTR 3′ UTR in our vectors does not include the poly U or G regulatory regions described by Baudouin-Legros et al.—so differences in post-transcriptional stability mediated by the 3′UTR appear moot. Lastly, the natural CFTR cDNA has a partial Kozak sequence (agACCatg) whereas a full Kozak sequence (CCACCatg) was included in the design of CO-CFTR.

TABLE 3
Features of CFTR expression plasmids

| | | | | alternative | | | | | |
|---|---|---|---|---|---|---|---|---|---|
| | | | | splice | | 5′ UTR | 3′ UTR | codon | |
| | | promoter | intron | acceptors † | CPGs | (bp) †† | (bp) †† | optimization | Kozak |
| natural | pCMVCFTR | CMV | CMV intron A | 2 | 58 | 10 | 41 | no | partial |
| cDNA | | | | | | | | | |
| natural | pUCF | UbC | UbC 1^{st} intron | 2 | 58 | 10 | 41 | no | partial |
| cDNA | | | | | | | | | |
| synthetic | pUCF2 | UbC | UbC 1^{st} intron | 0 | 1 | 0 | 0 | yes | full |
| CO- | | | | | | | | | |
| CFTR | | | | | | | | | |

† evaluation of slice acceptor consensus sequence in first 300 coding sequences.
†† refers to number of bp of natural CFTR cDNA found in 5′ and 3′ UTRs.

### Example 3

### Evaluation of Expression Vector Encoding Synthetic hCFTR DNA

pUCF2 was dosed intranasally (IN) into Balb/C mice and lungs were harvested at days 2 and 14 for evaluation of CFTR mRNA. As shown in FIG. 1, pUCF2 generated a hCFTR/mCFTR ratio on day 2 of 9.8% (+/−15%, SD) which fell to 0.72% (+/−0.67%) on day 14. The mean day 2 signal for pUCF2 is comparable to pCMVCFTR and likely is achieving a biologically significant level of CFTR expression, with a hCFTR/mCFTR ratio >5-6%. Although CFTR mRNA expression was not maintained, the day 14 hCFTR/mCFTR ratios for pUCF2 were significantly higher than for pCMVCFTR and pUCF, which is a first step in prolonging CFTR expression. Interestingly, the day 2 expression for pUCF2 was considerably higher (35-fold) than for pUCF, which appears related to CO-CFTR since both plasmids are nearly otherwise identical. This finding suggests that CO-CFTR cDNA is transcribed and/or processed more efficiently than natural CFTR cDNA in the context of this vector.

### Example 4

### Vectors Encoding Synthetic hCFTR Sequence Produce Enhanced hCFTR Protein by IP/Western Blot Analysis

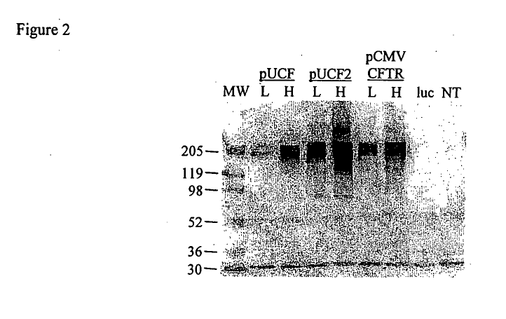

It is appreciated that CFTR protein production mediated by pUCF and pUCF2 likely involve differences in both transcriptional and translational efficiency. To directly compare these constructs, CFTR-negative HEK293 cells were transfected with lipofectamine and either pUCF or pUCF2. To minimize differences in transfection efficiency between plates, each liposome/DNA transfection mixture contained an equal total amount of plasmid DNA but differing amounts of test plasmid, an equal amount of luciferase plasmid (10 ng, to assess transfection efficiency), and appropriate amounts of ‘filler’ plasmid (Bluescript). Two days after transfection, lysates were prepared, luciferase activity assessed, and an IP/Western for CFTR performed (FIG. 2). Band densities were quantified and normalized for modest differences in luciferase activity. The Western blot shows a 9-fold increase in CFTR protein in cells transfected with pUCF2 compared to pUCF. Moreover, the relative abundance of CFTR protein in these transfected cells fits well with the rank-order of CFTR mRNA on day 2 in transfected murine lung (FIG. 1), suggesting that these cell line studies may have some relevance for the in vivo lung gene transfer setting.

### Example 5

### Vectors Encoding Synthetic hCFTR Sequence Produce Enhanced hCFTR Protein by ELISA Analysis

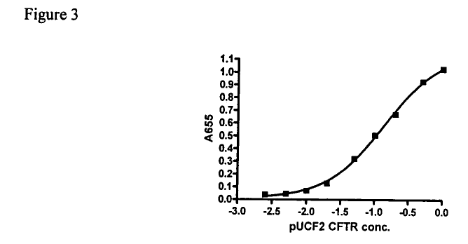

We have established an ELISA assay for hCFTR that incorporates anti-hCFTR Mab 1660 (R&D Systems) as capture and rabbit anti-hCFTR antibody (Santa Cruz, #10747) for detection. Mab 1660 detects a hCFTR epitope between codons 590-830 and rabbit polyclonal #10747 detects the N-terminus, amino acids 1-182, of human, rat, and mouse CFTR. The assay was optimized using lysates from HEK293 cells transfected with pUCF2 and a typical standard curve is shown in FIG. 3. By IP-Western, a 9-fold increase in hCFTR protein was detected in lysates from HEK293 cells transfected with pUCF2 compared to pUCF. By this ELISA, these lysates have a 19-fold increase in hCFTR protein, and by qRT-PCR analysis (for the 3 μg transfectants), there was a 39-fold increase in mRNA abundance.

### Example 6

### qRT-PCR Evaluation of hCFTR Expression

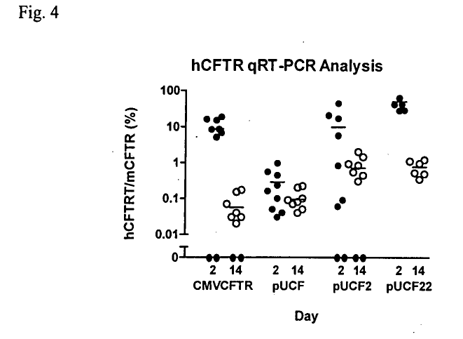

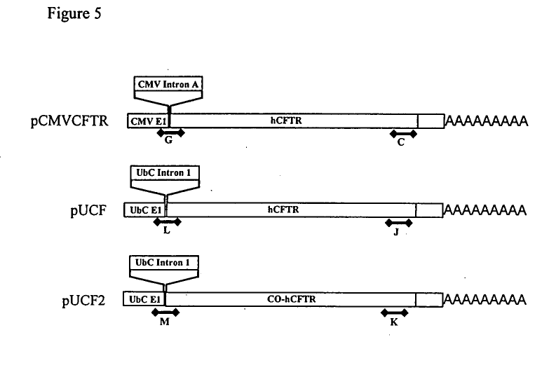

qRT-PCR data for hCFTR indicated that CO-CFTR (codon-optimized, CpG depleted except for one 3′ site, natural UTRs depleted, optimized Kozak sequence) generated 35-fold higher levels of hCFTR/mCFTR mRNA in murine lung at day 2 compared to natural CFTR cDNA in the identical plasmid (pUCF2 vs. pUCF, see FIG. 1) This improved day 2 result correlated well with evidence of enhanced CFTR protein expression (9-fold higher) at day 2 in transfected HEK293 cells by IP-Western analysis. However, the day 2 level of hCFTR/mCFTR mRNA expression in murine lung was not maintained, with the day 14 ratio falling to 0.72%. To compare CO-CFTR with a completely CpG depleted derivative (CO*-CFTR), the 3′ terminal CpG was removed and CO*-CFTR was subcloned in the pUL8 vector (pUCF22), which also introduces, for the first time, a clinically appropriate Km^{R} (kanamycin resistance) gene. To attempt to improve on expression levels, pUCF22 was dosed intratracheally at 200 μg rather than the prior intranasal 100 μg dose. As shown in FIG. 4, the day 2 hCFTR/mCFTR level increased to 50.6% (+/−27%, SD), but by day 14 had fallen to 0.77% (+/−0.36), comparable to the pUCF2 results.

Removal of the 3′ CpG in CO-CFTR did not improve persistence. The 5-fold improvement of hCFTR expression on day 2 for pUCF22 compared to pUCF2 can be explained by the difference in route of administration. As we have previously published, a 3-fold improvement in lung expression is expected comparing intratracheal and intranasal administration (Mol Therapy 8:936-947, 2003). In an intratracheal dose response study, there was little difference between 100 and 300 μg DNA doses (Mol Therapy 8:936-947, 2003).

As summarized in Table 4, it is apparent that the identical plasmid design encoding luciferase (e.g. pUL, pUL8) produces high level and maintained activity at day 14 whereas CFTR mRNA expression is extinguished, which could be due to:
a) a time-dependent decrease in CFTR mRNA stability;
b) a time-dependent decrease in the efficiency of CFTR mRNA transcription and/or aberrant splicing;
c) heterochromatin formation in the plasmid, extinguishing CFTR transcription, which could be due to chromatin encroachment from the prokaryotic backbone or direct heterochromatin formation in the eukaryotic cassette via de novo nucleosome formation and/or other chromatin structures;
d) selective loss of CFTR expression plasmids (whereas luciferase plasmids are evidently maintained in view of activity data).

TABLE 4
Relative Levels of Luciferase and CFTR Expression in CMV and UbC vectors†.

| | | Relative luciferase activity (%) | | hCFTR/mCFTR mRNA (%) | |
|---|---|---|---|---|---|
| Plasmid | | Day 2 | Day 14 | Day 2 | Day 14 (% of day 2) |
| CMV | pCMVluc‡ | 100 | 0 | | |
| | pCMVCFTR | | | 8.7 | 0.06 (0.7%) |
| UBC | pUL‡ | 100 | 412, 174 | | |
| | pUCF | | | 0.28 | 0.09 (32%) |
| | pUCF2 | | | 9.8 | 0.72 (7%) |
| | pUCF22 | | | 50.6 | 0.77 (1.5%) |

†All intranasal dosing of ~100 μg DNA, except pUCF22, which was an intratracheal dosing of ~200 μg DNA.
‡The luciferase activities (RLU/mg protein/μg DNA) of pCMVluc and pUL are essentially identical on day 2.

The mechanism(s) accounting for these results is currently undefined. It is possible that autoregulatory pathways exist that control CFTR mRNA expression. Although each CFTR plasmid had significantly reduced mRNA expression by day 14, both plasmids encoding codon-optimized and CpG-depleted CFTR had hCFTR/mCFTR levels about 35-fold higher than natural CFTR cDNA, suggesting that removal of CpGs, and/or the altered DNA and/or mRNA sequence, influenced the extinction process.

### Example 7

### Evaluation of Splicing Efficiency Reveals Presence of Premature Transcriptional Terminator

One possible mechanism that may influence CFTR mRNA expression is intron-mediated 5′ splicing. A detailed analysis of alternative splice acceptor sites in natural and CO-CFTR shows several 5′ sites present in natural but not CO-CFTR that result in out-of-frame transcripts. To assess intron splicing, hCFTR cDNA was evaluated by qRT-PCR analysis using primer sets bridging the desired donor-acceptor sequence as well as a 3′ primer set used previously in our hCFTR mRNA analysis (FIG. 4). HEK293 cells were transfected with either 0.75 or 3 μg of pCMVCFTR (our prior clinical trial plasmid), pUCF, or pUCF2, and cells were harvested at 2 days for mRNA analysis as well as CFTR protein expression by IP-Western. cDNA was generated using random primers and different CFTR transcript forms were detected by qRT-PCR using TaqMan probes. FIG. 5 illustrates the probe design to detect appropriately spliced (G,L,M) and total (C,J,K) hCFTR transcripts.

This transcript splicing analysis demonstrates some interesting and unexpected findings. Table 5 summarizes key results from this qRT-PCR analysis and the full data set are included in the Table 7 to permit detailed review of findings in this summary table.

TABLE 5
qRT-PCR Splice Analysis of hCFTR Transcripts in HEK293 Cells 2 Days After Transfection.

| | | 3′ | 5′ | R.E. 3′ | R.E. 5′ | | |
|---|---|---|---|---|---|---|---|
| | hGAPDH | hCFTR | hCFTR | hCFTR | hCFTR | Spliced (5′):Total | 5′/3′ hCFTR, |
| Sample No | Ct | Ct | Ct | % | % | (Spliced + Unspliced) (3′) | % |
| pUCF_075 | 16.294 | 19.92 | 19.595 | 0.90 | 1.13 | 1.3:1 | 125.27 |
| pUCF_3 | 17.016 | 19.654 | 18.591 | 1.79 | 3.75 | 2.1:1 | 208.93 |
| pUCF2_075 | 16.467 | 16.083 | 18.338 | 14.57 | 3.05 | 1:4.8 | 20.95 |
| pUCF2_3 | 16.438 | 13.8 | 15.876 | 69.50 | 16.48 | 1:4.2 | 23.72 |
| pCMV-CFTR_075 | 16.61 | 16.448 | 14.908 | 12.49 | 36.32 | 2.9:1 | 290.79 |
| pCMV-CFTR_3 | 16.363 | 14.416 | 13.2 | 43.05 | 100.00 | 2.3:1 | 232.30 |

The data for CO-CFTR (pUCF2) indicate that ˜23% of transcripts are appropriately spliced, whereas findings for natural CFTR expressed by either pUCF or pCMVCFTR transfectants generate non-logical results (bolded in table), with the 5′ signal (spliced) being more abundant than the 3′ signal (total). This can occur only if the 3′ primers and probes are NOT detecting the total population of transcripts. This phenomenon could occur if there is an alternative, ‘stealth’ pA site within the natural hCFTR cDNA. Interestingly, a sequence analysis of natural hCFTR cDNA for the ‘AATAAA’ transcription termination consensus sequence did turn up 1 match, at position 1100 of the open reading frame. Because of the codon-optimization of hCFTR in pUCF2, there are NO ‘AATAAA’ sequences in CO-CFTR.

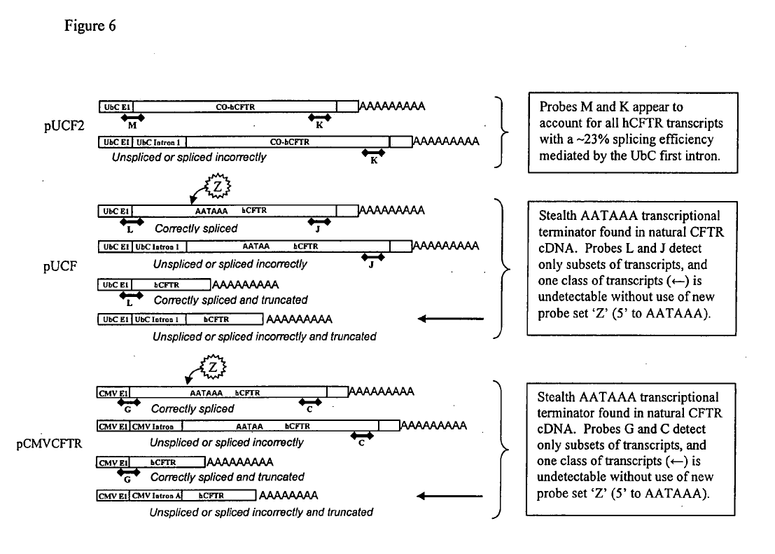

The data are consistent with the following working hypothesis (FIG. 6). Whereas pUCF2 appears to generate an understandable RT-PCR result with a ˜23% splicing efficiency, pUCF and pCMVCFTR appear to generate 4 classes of transcripts and a more complex picture.

As noted in FIG. 6, this analysis was continued using new probe set ‘Z’, which should be able to measure the entire set of proposed transcripts. For pCMVCFTR and pUCF, primer set Z should be more abundant than either C or J if this proposed scenario is correct. This analysis will permit the following to be determined from the relative efficiency (R.E. in Table 5) generated from these probe sets:

TABLE 6

| | % FULL LENGTH | |
|---|---|---|
| | TRANSCRIPTS | SPLICING EFFICIENCY |
| pUCF2 | defined by K | M/K |
| pUCF | J/Z | L/Z |
| pCMVCFTR | C/Z | G/Z |

Based on our data, it appears that a significant percentage of hCFTR transcripts generated by pUCF and pCMVCFTR are truncated, with quantification pending the ‘Z’ probe analysis. This finding may account, in part, for multiple findings, including: i) the significant increase in the 3′-based qRT-PCR hCFTR signal for pUCF2 compared to pUCF (35-fold increase at day 2 in mouse lung, 16-39-fold increase in HEK293 cells); and most importantly, ii) the difficulty in measuring hCFTR mRNA in various CF clinical trials, which often have employed 3′ primer sets.

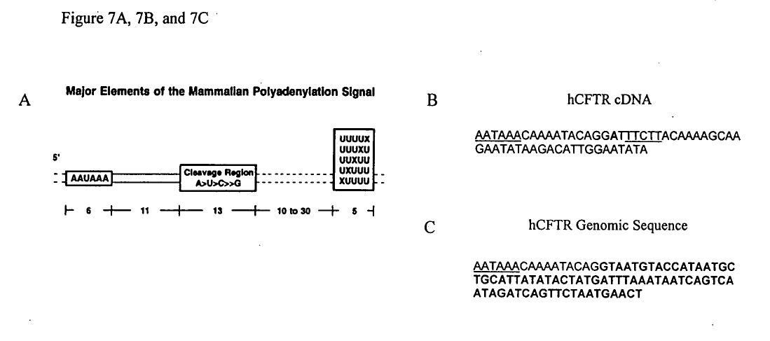

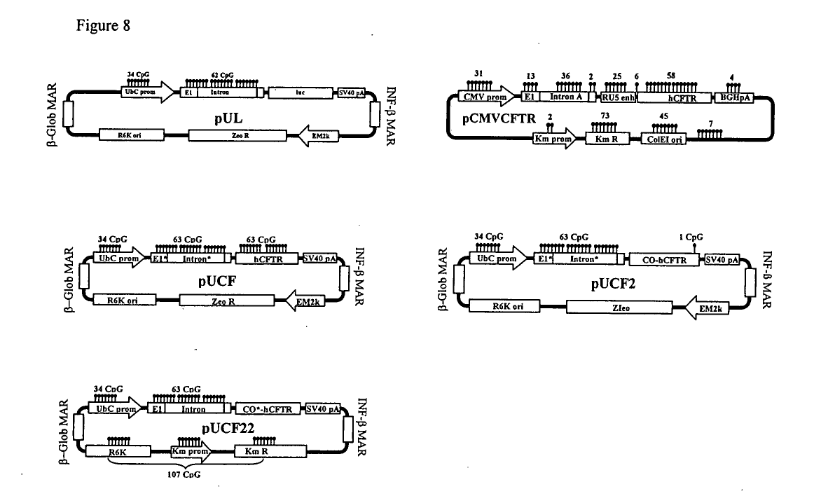

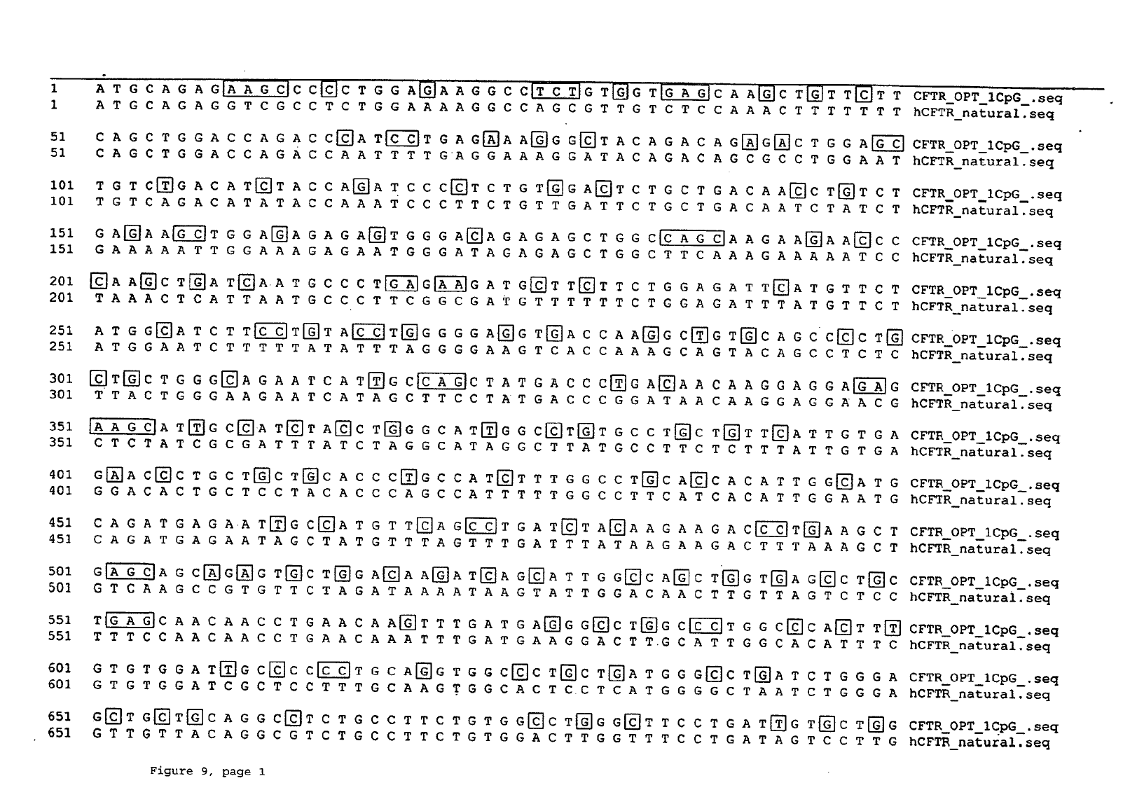

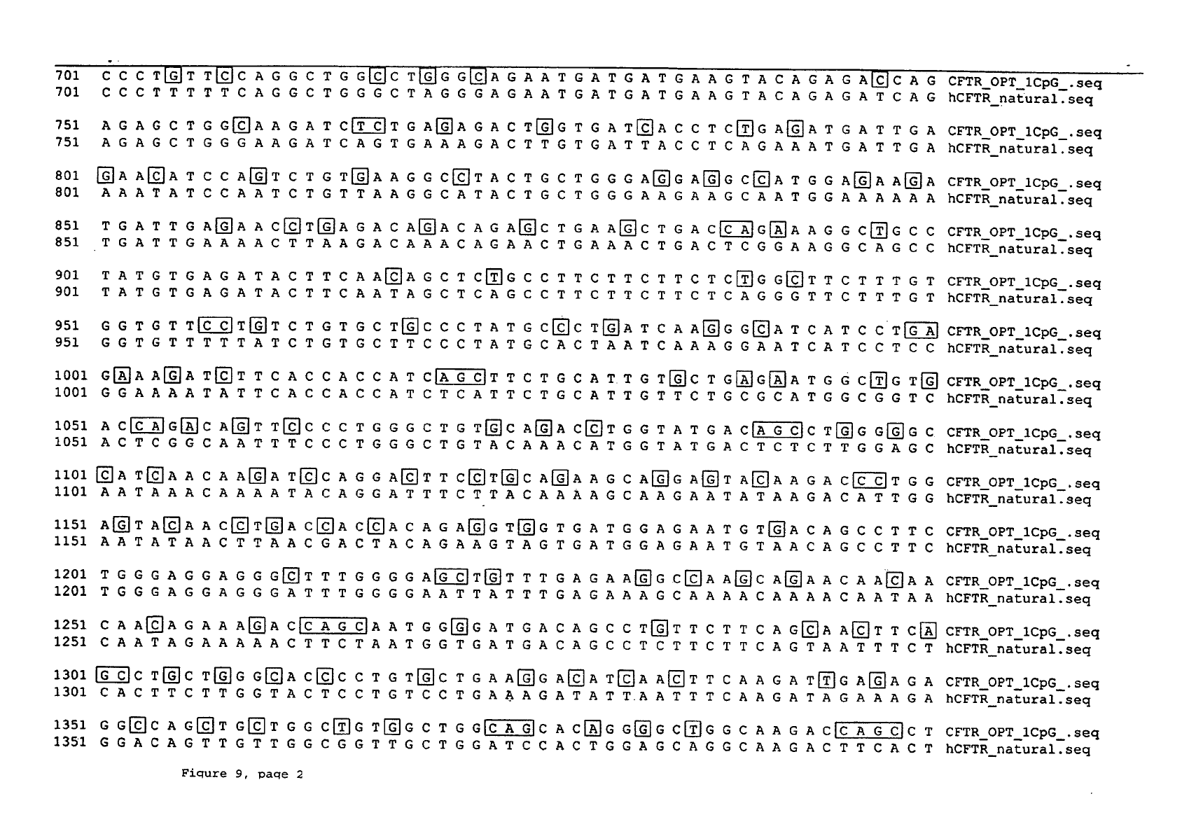

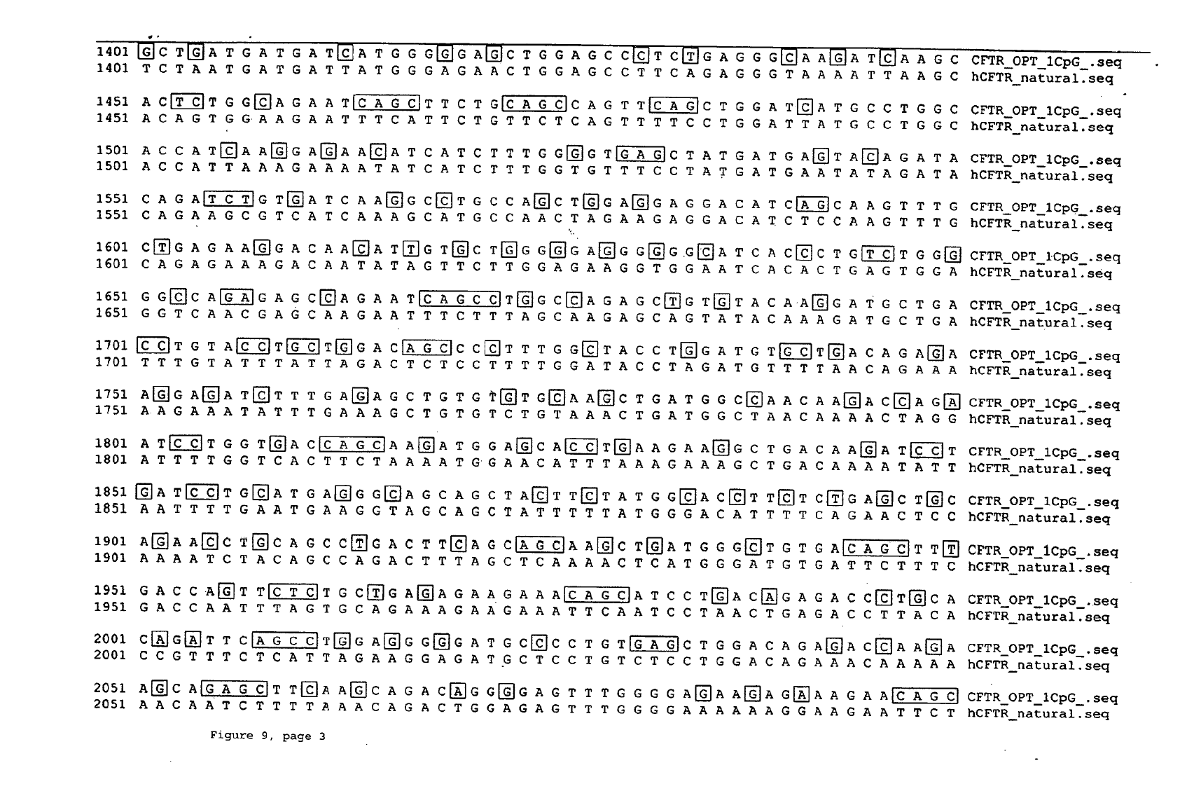

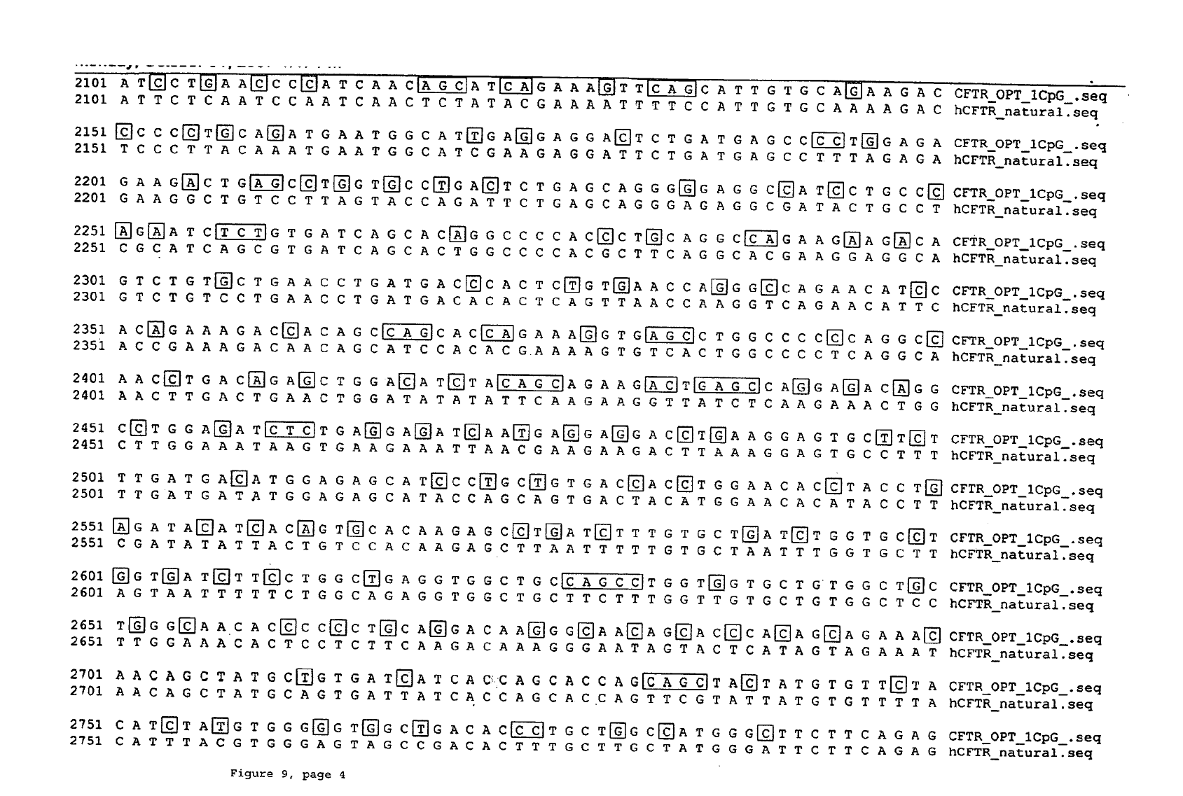

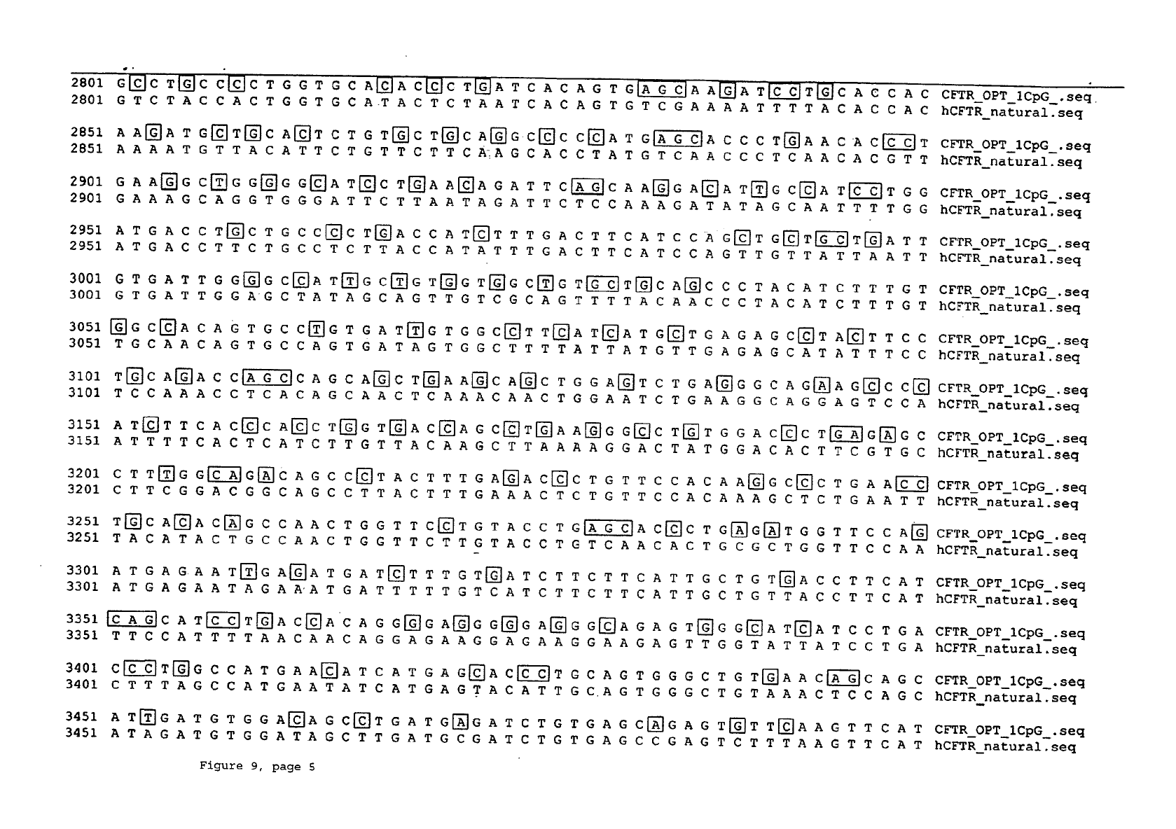

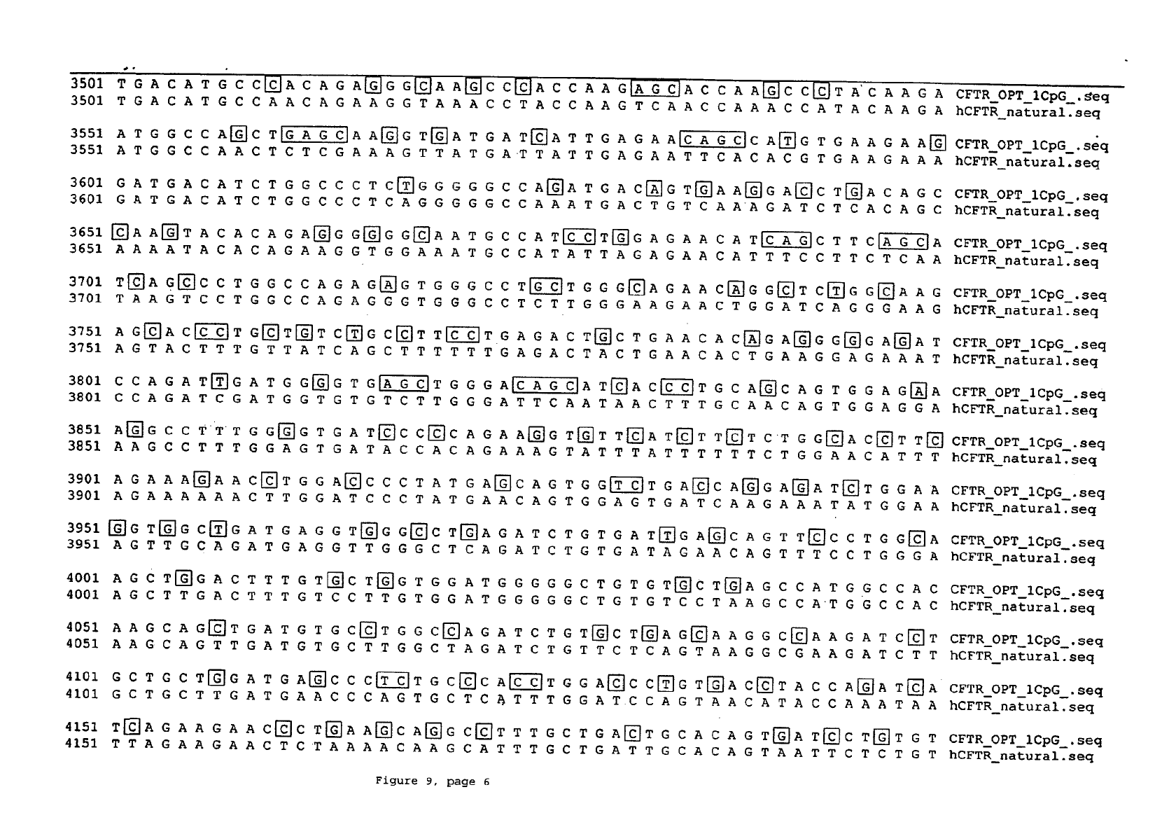

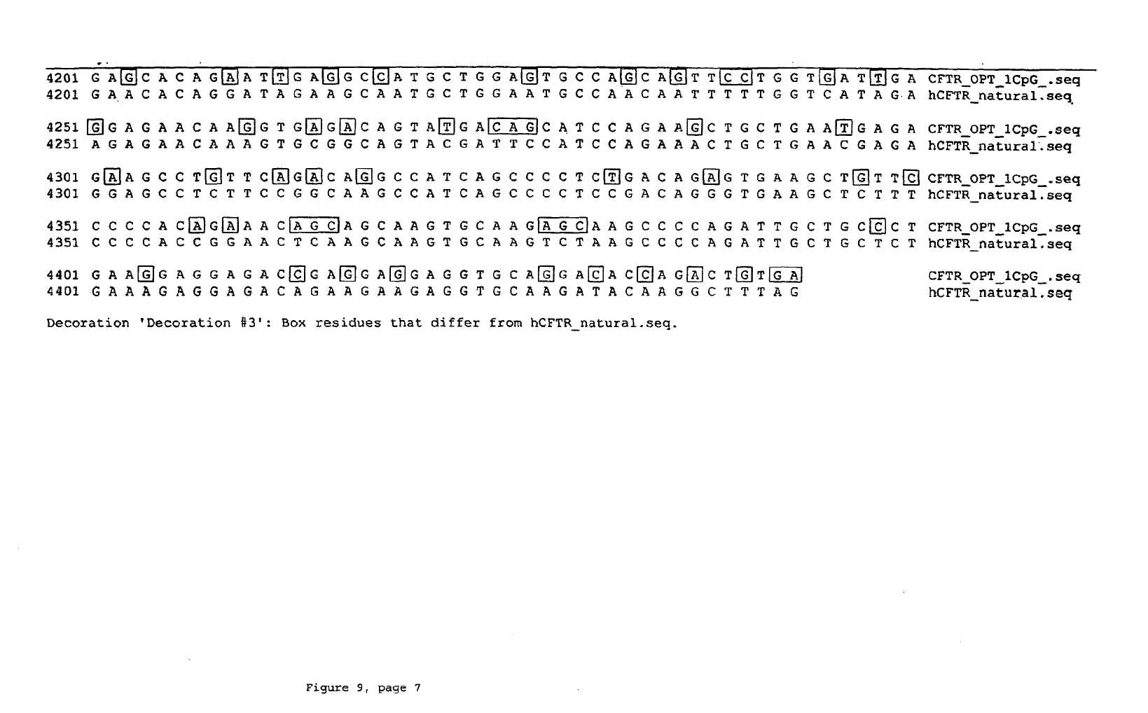

It is important to note that a DNA sequence analysis suggests that this putative transcriptional terminator is active in natural hCFTR cDNA but NOT in genomic DNA (FIG. 7), so that endogenous hCFTR would not be subject to premature truncation. As reported by Chen et al. (2), the following sequence motifs are usually present at transcriptional termination sites. Following ‘AAUAAA’ there typically is a uridine rich element (URE). The cleavage site typically occurs 11 to 24 bases downstream of ‘AAUAAA’, and the URE sequence occurs 10 to 30 bp after the cleavage site. In natural hCFTR cDNA, a URE motif is present at +14 after ‘AATAAA’, with a potential cleavage site at +12. In contrast, a URE site is not found downstream of ‘AATAAA’ in genomic hCFTR DNA (which is closely followed by intron 7), suggesting this potential termination motif is not functional. In summary, DNA sequence considerations suggest that this putative transcriptional termination site may be active in “natural” hCFTR cDNA but not in either genomic hCFTR or CO-hCFTR DNA.

If our pending ‘Z’ and ‘Q’ probe studies generate data as predicted, then there are several important implications of this splice analysis:
1. A stealth transcription terminator exists in natural CFTR cDNA that appears to be terminating the majority of transcripts produced by pCMVCFTR and pUCF;
2. In genomic hCFTR DNA, the putative ‘AATAAA’ pA site occurs in exon 7 and is closely followed by intron 7 sequences which do NOT have a URE motif, suggesting that it is NOT functional;
3. Presence of this stealth pA site may have reduced the ability to detect hCFTR mRNA in prior human CF clinical trials;
4. The codon-optimized hCFTR cDNA does not have this stealth transcription terminator sequence and mRNA levels in treated CF patients may not suffer from this limitation.

TABLE 7
5′ Splice Site Analysis of HEK293 Cells Transfected with pCMVCFTR, pUCF, and pUCF2.

| | | 3′ | 5′ | 3′ | 5′ | | 3′ | 5′ | | R.E. 5′ | 5′/3′ |
|---|---|---|---|---|---|---|---|---|---|---|---|
| | hGAPDH | hCFTR | hCFTR | hCFTR | hCFTR | Cali- | hCFTR | hCFTR | R.E. 3′ | hCFTR | hCFTR, |
| Sample No | Ct | Ct | Ct | ΔCt | ΔCt | brator | ΔΔCt | ΔΔCt | hCFTR % | % | % |
| pUCF_075 | 16.294 | 19.92 | 19.595 | 3.626 | 3.301 | −3.163 | 6.789 | 6.464 | 0.90 | 1.13 | 125.27 |
| pUCF_3 | 17.016 | 19.654 | 18.591 | 2.638 | 1.575 | | 5.801 | 4.738 | 1.79 | 3.75 | 208.93 |
| pUCF2_075 | 16.467 | 16.083 | 18.338 | −0.384 | 1.871 | | 2.779 | 5.034 | 14.57 | 3.05 | 20.95 |
| pUCF2_3 | 16.438 | 13.8 | 15.876 | −2.638 | −0.562 | | 0.525 | 2.601 | 69.50 | 16.48 | 23.72 |
| pCMV- | 16.61 | 16.448 | 14.908 | −0.162 | −1.702 | | 3.001 | 1.461 | 12.49 | 36.32 | 290.79 |
| CFTR_075 | | | | | | | | | | | |
| pCMV- | 16.363 | 14.416 | 13.2 | −1.947 | −3.163 | | 1.216 | 0 | 43.05 | 100.00 | 232.30 |
| CFTR_3 | | | | | | | | | | | |

HEK293 cells were transfected with either 0.75 or 3 μg of each plasmid (along with ‘blank’ irrelevant plasmid so that the total DNA/lipofectamine ratios were the same) and harvested 2 days later. qRT-PCR was performed using TaqMan chemistry and FAM-TAMRA labeled probes. Each reaction was performed as quadruplicate replica. In some cases, outrangers were removed and analysis was performed on three point average values. The Cts for 5′ hCFTR and 3′ hCFTR were estimated using a 0.4 threshold value. The Cts for hGAPDH were estimated using a 0.2 threshold value. Vector contamination was insignificant. R.E., relative expression.

HEK293 cells were transfected with either 0.75 or 3 μg of either pCMVCFTR or pUCF. Cells were harvested 2 days later for RNA preparation and qRT-PCR analysis using validated primer sets J and Z6. Shown in Table 8 is a summary of these findings. Approximately 27-38% of these vector-derived natural hCFTR transcripts appear to demonstrate premature truncation. Primer-probe validation experiments show that qPCR Δ Ct accuracy is in the +/−7% range. So, the observed differences in Z6 and J amplification cannot be explained by different amplification efficiencies. Otherwise, note that HEK293 cells do not transcribe detectable endogenous hCFTR mRNA (saline controls were negative), so all signals are derived from the vector. These results are consistent with the known ability of natural hCFTR cDNA to produce functional hCFTR protein, but underscore the potential inefficiency of this cDNA. Of note, the CO-CFTR and the CO*-CFTR constructs do not have this transcriptional termination sequence.

TABLE 8
Transcriptional Truncation Analysis of HEK293 Cells Transfected with Natural hCFTR Vectors.

| | Avg. J | Avg. Z6 | J − Z6 | Adj. Z6 | J − Adj. Z6 | Z6/J R.E. | Truncated/ |
|---|---|---|---|---|---|---|---|
| Vector | (Ct) | (Ct) | (Ct) | (Ct) | (Ct) | Ratio† | Total§ |
| pCMV CFTR (0.75 μg) | 19.448 | 18.536 | 0.912 | 18.75532 | 0.692683 | 1.62 | 38% |
| pCMV CFTR (3 μg) | 17.99 | 17.353 | 0.637 | 17.53727 | 0.452733 | 1.37 | 27% |
| pUCF (0.75 μg) | 22.378 | 21.453 | 0.925 | 21.75874 | 0.619258 | 1.54 | 35% |
| pUCF (3 μg) | 24.591 | 22.662 | 1.929 | 23.00356 | 1.587437 | 3.01‡ | |

†relative efficiency ratio is calculated: 2\hat{ }(J − Adj. Z)
‡data from this transfection will not be considered based on evidence of RNA degradation from mGAPDH primers
§truncation percentage = 1 − 2\hat{ }(Adj. Z − J)

The next step was to determine if natural hCFTR cDNA prematurely truncates in transfected mouse lungs (Table 9). To conduct this analysis, four lung samples with the highest level of hCFTR mRNA expression (based on 3′ J primers) were selected; all of these samples were from the day 2 pCMVCFTR group, which had a ˜30-fold higher level of hCFTR/mCFTR expression than pUCF transfectants. Based on mGAPDH primers, the M5 sample had ˜2-4-fold less cDNA than the other samples, which otherwise were comparable. All samples demonstrated evidence of premature truncation, ranging from 14% to 62%. The reason for the lower value for sample M5 is unclear, although its lower mGAPDH signal raises a question about sample integrity (versus smaller sample). In summary, studies from HEK293 cells and mouse lungs dosed with pCMVCFTR both demonstrate evidence of premature transcriptional truncation for natural hCFTR cDNA. Similar data in both cells and animals strongly support this conclusion.

TABLE 9
Transcriptional Truncation Analysis of Balb/c Mouse
Lungs Dosed with Natural hCFTR Vector pCMVCFTR.

| pCMVCFTR | | | | | | | | |
|---|---|---|---|---|---|---|---|---|
| dosed animals (day | mGAPDH | | | | Adj. Z6 | J − Adj. Z6 | Z6/J R.E. | Truncated/ |
| 2) | Ct | J Ct | Z6 Ct | J − Z6 Ct | Ct | Ct | Ratio† | Total‡ |
| M5 | 22.052 | 33.511 | 32.649 | 0.862 | 33.286 | 0.2245 | 1.1684 | 14.4% |
| M6 | 20.206 | 32.434 | 30.462 | 1.972 | 31.035 | 1.3993 | 2.6378 | 62.1% |
| M8 | 20.905 | 32.147 | 30.699 | 1.448 | 31.279 | 0.8683 | 1.8255 | 45.2% |
| M11 | 20.924 | 32.748 | 30.855 | 1.893 | 31.439 | 1.3087 | 2.4772 | 59.6% |

†relative efficiency ratio is calculated: 2\hat{ }(J − Adj. Z)
‡truncation percentage = 1 − 2\hat{ }(Adj. Z − J)

### CONCLUSIONS

A truncation analysis of hCFTR mRNA transcribed from the pCMVCFTR vector (natural hCFTR cDNA) in both HEK293 cells and the mouse lung demonstrates evidence of premature truncation. The percentage of transcripts that are truncated range from 27-38% in HEK293 cells and 14-62% in the mouse lung.

Premature transcriptional termination may only partially account for the 35-fold difference in hCFTR/mCFTR mRNA expression in the mouse lung on day 2 when comparing pUCF2 (CO*-hCFTR) to pUCF (natural hCFTR cDNA). Other factors may be important, including potential differences in nucleosome formation within the plasmid, CpG depletion of the transgene, and potential improvement in mRNA half-life due to changes in the primary ribonucleotide sequence. The 9- to 19-fold improvement in hCFTR protein in HEK293 cells transfected with pUCF2 compared to pUCF may be accounted for by improved hCFTR mRNA abundance, although codon-optimization also may be important.

### REFERENCES

The disclosure of each reference cited is expressly incorporated herein.
1. Baudouin-Legros M, Hinzpeter A, Jaulmes A, Brouillard F, Costes B, Fanen P, and Edelman A. Cell-specific posttranscriptional regulation of CFTR gene expression via influence of MAPK cascades on 3′UTR part of transcripts. Am J Physiol Cell Physiol. 289(5), C1240-1250, 2005.
2. Chen F, MacDonald CC, and Wilusz J. Cleavage site determinants in the mammalian polyadenylation signal. Nucleic Acids Res. 23(14):2614-2620, 1995.
3. Griesenbach U, Geddes D M, Alton E W. Gene therapy progress and prospects: cystic fibrosis. Gene Ther. 2006 July; 13(14):1061-7.
4. Griesenbach U, Geddes D M, Alton E W. Gene therapy for cystic fibrosis: an example for lung gene therapy. Gene Ther. 2004 October; 11 Suppl 1:S43-50.
5. Davis P B, Cooper M J. Vectors for airway gene delivery. AAPS J. 2007 Jan. 19; 9(1):E11-7.
6. Davis P B. Cystic fibrosis since 1938. Am J Respir Crit Care Med. 2006 Mar. 1; 173(5):475-82. Epub 2005 Aug. 26.
7. Konstan M W, Davis P B, Wagener J S, Hilliard K A, Stern R C, Milgram L J, Kowalczyk T H, Hyatt S L, Fink T L, Gedeon C R, Oette S M, Payne J M, Muhammad O, Ziady A G, Moen R C, Cooper M J. Compacted DNA nanoparticles administered to the nasal mucosa of cystic fibrosis subjects are safe and demonstrate partial to complete cystic fibrosis transmembrane regulator reconstitution. Hum Gene Ther. 2004 December; 15(12):1255-69.
8. Ziady A G, Gedeon C R, Miller T, Quan W, Payne J M, Hyatt S L, Fink T L, Muhammad O, Oette S, Kowalczyk T, Pasumarthy M K, Moen R C, Cooper M J, and Davis P B: Transfection of airway epithelium by stable PEGylated poly-L-lysine DNA nanoparticles In Vivo. Mol Ther, 8:936-947, 2003.
9. Ziady A G, Gedeon C R, Muhammad O, Oette S M, Fink T L, Quan W, Kowalczyk T H, Hyatt S L, Peischl A, Seng J E, Moen R C, Cooper M J, and Davis P B: Minimal toxicity of stabilized compacted DNA nanoparticles in the murine lung. Mol Ther, 8:948-956, 2003.
10. Liu G, Li D, Pasumarthy M K, Kowalczyk T H, Gedeon C, Hyatt S, Payne J M, Miller T J, Brunovskis P, Moen R C, Hanson R W, and Cooper M J: Nanoparticles of compacted DNA transfect post-mitotic cells. J Biol Chem, 278:32578-32586, 2003.

We claim:

1. A composition comprising a nucleic acid molecule comprising a sequence as shown in SEQ ID NO: 1 or 2 (DNA) or SEQ ID NO: 3 or 4 (RNA).

2. The composition of claim 1 wherein the sequence is SEQ ID NO: 1.

3. The composition of claim 1 wherein the sequence is SEQ ID NO: 2.

4. The composition of claim 1 wherein the sequence is SEQ ID NO: 3.

5. The composition of claim 1 wherein the sequence is SEQ ID NO: 4.

6. The composition of claim 1 wherein the nucleic acid molecule is a non-viral vector.

7. The composition of claim 1 wherein the nucleic acid molecule is a viral vector.

8. The composition of claim 1 wherein the composition comprises cells in which the nucleic acid molecule is expressed.

9. The composition of claim 1 further comprising mammals comprising cells in which the nucleic acid molecule expresses.

10. A method of producing hCFTR-encoding mRNA and hCFTR protein comprising:
introducing a composition comprising a nucleic acid molecule comprising a sequence as shown in SEQ ID NO: 1 or 2 (DNA) or SEQ ID NO: 3 or 4 (RNA) into mammalian cells, whereby the cells express hCFTR-encoding mRNA and hCFTR protein.

11. The method of claim 10 wherein the cells are human cells.

12. The method of claim 10 wherein the cells are lung cells.

13. The method of claim 10 wherein the cells are in a human lung.

14. The method of claim 10 wherein the cells are lung cells in an animal model of Cystic Fibrosis.

15. The method of claim 13 wherein the human lung is in a Cystic Fibrosis patient.

16. The method of claim 15 wherein the composition is introduced via an aerosol.

17. The method of claim 15 wherein the nucleic acid molecule is compacted in particles with a polycation, wherein the particles are unimolecular with respect to nucleic acid.

18. The method of claim 11 wherein the human cells are in a Cystic Fibrosis patient.

19. The method of claim 18 wherein the nucleic acid molecule is compacted in particles with a polycation, wherein the particles are unimolecular with respect to nucleic acid.

20. The method of claim 19 wherein the composition is introduced via an aerosol.

21. The method of claim 19 wherein the composition is introduced intravenously.

22. The method of claim 19 wherein the composition is introduced via Endoscopic Retrograde Cholangiopancreatography (ERCP).

23. The method of claim 19 wherein the composition is introduced directly to the pancreas.

24. The method of claim 19 wherein the composition is introduced in utero to a fetus.

25. A method of producing hCFTR-encoding mRNA and hCFTR protein comprising:
introducing a composition comprising a nucleic acid molecule comprising a sequence as shown in SEQ ID NO: 1 or 2 (DNA) or SEQ ID NO: 3 or 4 (RNA) into human lung cells in a human Cystic Fibrosis patient via an aerosol, wherein the nucleic acid molecule is compacted in particles with a polycation, wherein the particles are unimolecular with respect to nucleic acid, whereby the cells express hCFTR-encoding mRNA and hCFTR protein.

26. A method to increase the expression of an mRNA or protein from a cDNA molecule, comprising:
inspecting the cDNA molecule to ascertain the presence of a premature transcription termination signal;
eliminating the premature transcription termination signal of the cDNA molecule without altering its encoded amino acid sequence;
introducing the cDNA into a cell whereby it is expressed.

27. The method of claim 26 wherein the cDNA encodes CFTR.
